(19) United States
(12) Patent Application Publication
(10) Pub. No.: US 2022/0157424 A1
(43) Pub. Date: May 19, 2022

(54) SYSTEM AND METHOD FOR EXAMINING TEST SAMPLES ON A VIRTUAL PLATFORM

(71) Applicant: EONBT Private Limited, Kanpur (IN)

(72) Inventors: Kawaldeep Arora, NEW DELHI (IN); Ankur Singh Bist, Bijnor (IN); Saksham Chaurasia, Gorakhpur (IN); Roshan Prakash, Udupi (IN); Vikas Pandey, NEW DELHI (IN); Suraz Kottakki, Danang (VN); Sharada Dutt Yadav, NEW DELHI (IN)

(21) Appl. No.: 17/207,843

(22) Filed: Mar. 22, 2021

(30) Foreign Application Priority Data
Nov. 16, 2020 (IN) 202011049830

(51) Int. Cl. G16H 15/00; G16H 10/60; H04L 9/32; G06K 9/00

(52) U.S. Cl. CPC G16H 15/00; G16H 10/60; H04L 9/3231; G06K 2209/01; H04L 2209/38; H04L 2209/88; G06K 9/00442

(57) ABSTRACT

Disclosed is a system for authentication of medical certificates using a distributed ledger (100) including a code generation module (110), a medical certificate generation module (120), a medical certificate authentication module (130), an identity authentication module (150), and a central server (160). Further is disclosed a method for authentication of medical certificates using a distributed ledger (200) utilising the above mentioned components of the system for authentication of medical certificates using a distributed ledger (100) along with a user module (170).

### FIELD OF INVENTION

The present embodiment generally relates to the field of authenticating medical equipment, tests, and certificates. More particularly, the present invention relates to a system and a method for authentication of medical certificates utilising a distributed ledger of information.

### BACKGROUND OF THE INVENTION

This section is intended only to provide background information pertaining to the similar field of the present invention, and may be used only to enhance the understanding of the present invention and not as admissions of prior art.

Although falling ill and/or having medical ailments is common, it is not uncommon for a person undergoing an ailment to require a medical certificate.

Medical certificates are a written statement from a physician or another medically qualified health care provider which attests to the result of a medical examination of a person. It can serve as a sick note (documentation that an employee is unfit for work) or evidence of a health condition.

Medical certificates are often required to certify that someone is free of contagious diseases, drug addiction, mental illness, or other health issues. Health criteria are often required when making an application for something, such as an eye examination to get a driver's license. Additionally, they may also be utilised to certify that someone has a contagious diseases, drug addiction, mental illness, or other health issues, for legal or professional purposes.

Conventionally, medical certificates can be attained from a medical professional, who provides a diagnosis on the basis of check-ups or tests. However, it often takes up more of a person's time to specifically get a medical certificate since medical professionals are more prone to providing prescriptions outlining the medicines, tests and other requirements to be taken care of by the person with an ailment.

In such cases, specifically procuring a medical certificate may become hassle-some since it would require additional time from the person having the ailment as well as the medical professional.

Additionally, since medical certificates are usually printed on paper and physically provided, they often get misplaced, lost, or destroyed. There is also a possibility of duplicate or forged medical certificates, which are untrue and may provide false information pertaining to a person's health. This activity completely derails the utilisation and necessity of procuring medical certificates because forged certificates can be untrue.

Even if the medical certificates are digitised, the records may be altered for personal purposes and the information pertaining to the medical certificates can be forged, in a manner that such a chance may not be identified, or be untraceable to the person altering the records.

Therefore, in light of the above-mentioned limitations of the existing systems and methods of dispensing medical certificates, there exists a need of a system and method for authentication of medical certificates. More particularly, there exists a need to eliminate traditional systems and methods of dispensing medical certificates and introduce a system and method for authentication of medical certificates using a distributed ledger of information which assists in generating, encoding and authenticating medical certificates on the basis of a medical diagnosis.

Further, there exists a need of a system and method for authentication of medical certificates using a distributed ledger which simplifies the process for the medical professional to generate medical certificates, while also simplifying the process for the person to procure medical certificates.

Additionally, there exists a need of a system and method for authentication of medical certificates using a distributed ledger which utilises distributed ledger technology or block-chain technology to store all medical certificate data, as well as any changes being made to the same in real-time.

### SUMMARY OF INVENTION

In light of the limitations of the existing conventional systems as discussed above, it is evident that there arises a need of a system and method for authentication of medical certificates using a distributed ledger, wherein an encoding is placed on the generated medical certificates, the information regarding the encoding being stored onto the distributed ledger, which assists in authenticating and/or validating the medical certificates, while allowing a simplified process of generating and procuring medical certificates for substantially overcoming the above-mentioned limitations.

The present disclosure portrays a code generation module (110), a medical certificate generation module (120), a medical certificate authentication module (130), an identity authentication module (150), a central server (160), and a user module (170), making it a time efficient and appropriate process for ensuring generation and authentication of medical certificates, such that the medical certificates are being generated by authenticated persons, ensuring an appropriate and time-efficient distribution of generated medical certificates, and an appropriate authentication of medical certificates as applicable.

Another object of the present disclosure is to incorporate a unique identity code onto every medical certificate so as to ensure distribution of only authentic medical certificates, thereby reducing the deception caused by forged medical certificates which provide untrue information pertaining to the health and fitness of a person. Yet another object of the present disclosure is to incorporate a distributed ledger of information which authenticates the medical certificate by revisiting unique encoded information being affixed onto the medical certificate during generation of the same, the unique encoded information having details of the medical certificate in order to verify the authenticity of the medical certificates.

Another object of the present invention is to provide an identity authentication module utilising distributed ledger technology which allows the input of identity documents and utilises optical character recognition in order to understand the same, and verify a person's identity from governmental databases. Yet another object of the present invention is to provide the identity authentication module to have a face match and a liveliness test in order to ensure that the identity of the person generating, accessing or authenticating medical certificates is appropriate in order to reduce the wrongful forgery of the medical certificates.

Yet another object of the present invention is to integrate all above disclosed modules and technologies into the system and method for authentication of medical certificates using a distributed ledger which simplifies the process for the medical professional to generate medical certificates, while also simplifying the process for the person to procure medical certificates.

Another object of the present invention is to provide a system and method for authentication of medical certificates using a distributed ledger which utilises distributed ledger technology or block-chain technology to store all medical certificate data, as well as any changes being made to the same in real-time.

In an aspect, embodiments of the present disclosure provide a system for authentication of medical certificates using a distributed ledger (100) including a code generation module (110), a medical certificate generation module (120), a medical certificate authentication module (130), an identity authentication module (150), a central server (160), and a user module (170).

As used herein, a user connects with the system for authentication of medical certificates using a distributed ledger (100) via a user module (170). Further, the user module (170) is communicably coupled to the central server (160), which in-turn connects the user module (170) to the system for authentication of medical certificates using a distributed ledger (100). The code generation module (110), the medical certificate generation module (120), the medical certificate authentication module (130), and the identity authentication module (150), are all communicably coupled with the block-chain module (140), as well as each other. Additionally, the user connects to the block-chain module (140) using the user module (170) via the central server (160).

### BRIEF DESCRIPTION OF THE DRAWINGS

Other objects, features, and advantages of the embodiment will be apparent from the following description when read with reference to the accompanying drawings. In the drawings, wherein like reference numerals denote corresponding parts throughout the several views:

FIG. 1 illustrates a block diagram depicting a system for authentication of medical certificates using a distributed ledger (100), in accordance with an embodiment of the present disclosure.

FIG. 2 illustrates a process flow diagram depicting a method for authentication of medical certificates using a distributed ledger (200), in accordance with an embodiment of the present disclosure.

FIG. 3 illustrates a process flow diagram depicting a method for generating unique codes for medical certificates using a distributed ledger (300), in accordance with an embodiment of the present disclosure.

FIG. 4 illustrates a process flow diagram depicting a method for authentication of medical certificates (400) by the system for authentication of medical certificates using a distributed ledger (100), in accordance with an embodiment of the present disclosure.

### DETAILED DESCRIPTION OF THE PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Throughout the present disclosure the term “a system for authentication of medical certificates using a distributed ledger (100)” refers to a system which provides generation of a unique code on the basis of a person's medical diagnosis, generation of a medical certificate affixed with the unique code, distribution of the medical certificate, authentication of the medical certificate, and authentication of a user's identity. In light of the present disclosure, the system for authentication of medical certificates using a distributed ledger (100) may be utilised for appropriate generation, distribution and authentication of medical certificates such that forgery of medical certificates is reduced and ultimately eliminated, and only true information is provided in the issued medical certificates.

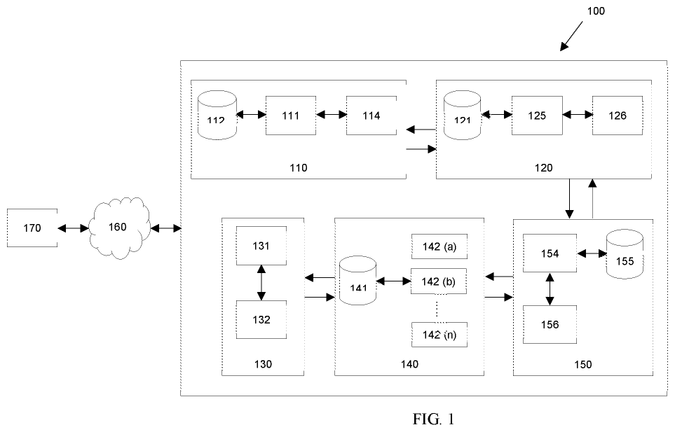

Referring to FIG. 1 illustrates a block diagram depicting a system for authentication of medical certificates using a distributed ledger (100), in accordance with an embodiment of the present disclosure. Furthermore, the system for authentication of medical certificates using a distributed ledger (100) comprises of a code generation module (110), a medical certificate generation module (120), a medical certificate authentication module (130), an identity authentication module (150), a central server (160), and a user module (170); and is utilised by a user via the user module (170) which is communicable coupled to the system for authentication of medical certificates using a distributed ledger (100) through the central server (160).

The code generation module (110) of the system for authentication of medical certificates using a distributed ledger (100) includes an encryptor (111), a plurality of coded information (112), and a printer (114) communicable coupled together. The plurality of coded information (112) entails all the information regarding the medical certificate, patient history and ailment details, symptoms, test results as well as the diagnostics. The plurality of coded information (112) is encoded by the encryptor (111) into a unique code. The encryptor (111) codes the plurality of coded information (112) into a unique code by utilising an encryption key (113). The unique code then gets placed on the medical certificate by the printer (114).

The medical certificate generation module (120) includes an information module (121), an input module (125), and an intelligence module (126). The information module (121) further includes medical professional information (122), patient information (123), and ailment information (124); and refers to all the information pertaining to and required to generate the medical certificate. The input module (125) is communicably coupled to the user module (170), wherein the medical practitioner can input all relevant information onto the system for authentication of medical certificates using a distributed ledger (100). Additionally, the intelligence module (126) verifies that the information pertaining to the medical certificate is linked to the unique code generated by the code generation module (110).

Further, the medical certificate authentication module (130) of the system for authentication of medical certificates using a distributed ledger (100) includes a code reader (131), and a scanning module (132). The code reader (131) may pertain to a camera or a scanner which can read the unique code generated by the code generation module (110), and the scanning module (132) captures all the information stored in the unique code, scans it, and converts it into decipherable text.

The unique code generated by the code generation module (110) is a unique encrypted graphic code. Further, the unique code might be a bar code, a QR code, a stamp, a symbol, a cipher, or any other unique encrypted graphic code.

The medical certificate can only be authenticated by an authorized personnel via the medical certificate authentication module (130). If a wrongful medical certificate is scanned, the medical certificate authentication module (130) of the system for authentication of medical certificates using a distributed ledger (100) alerts the authorities to forgery of the medical certificate as per predefined protocol.

Herein, predefined protocol may include flashing alert messages onto communication device screens of authorities, devices including but not limited to pagers, computers, mobiles, smart watches, laptops and tablets; sending urgent emails to the authorities on designated email addresses, and/or calling the authorities on designated numbers with a defined pre-recorded message in order to alert the authorities regarding forgery. When forgery in a medical certificate is observed, a separate team may investigate the reality and take necessary actions based thereupon.

The code generation module (110), the medical certificate generation module (120), and the medical certificate authentication module (130) are communicably coupled to the block-chain module (140). The block-chain module (140) of the system for authentication of medical certificates using a distributed ledger (100) includes a distributed ledger database (141), and a plurality of nodes (142a, 142b, 142c, . . . , 142n), wherein multiple different sets of information are stored separately, along with a timestamp of when the information was inserted or changed, and a digital signature of the person who inserted or changed the information. The distributed ledger database (141) stores the information regarding the position of the plurality of nodes (142a, 142b, 142c, . . . , 142n).

Additionally, only an authorised person is allowed to insert or change the information in the distributed ledger database (141).

The identity authentication module (150) includes an identity module (154), an identity database (155), and an optical character recognition module (156). The identity module (154) further includes a document module (151), a face match test (152), and a liveliness test (153), and is responsible for identifying the person using the system for authentication of medical certificates using a distributed ledger (100). Furthermore, the identity database (155) contains a plurality of information pertaining to the identity of people, and the optical character recognition module (156) is facilitated to extract information from documents.

The authentication of a user's identity may be done by a plurality of identity documents, a face match or a liveliness test. The user may upload documents onto the identity authentication module (150) via the user module (170). Once the documents are provided to the identity authentication module (150), they are understood by the document module (151) which assists the optical character recognition module (156) to understand the documents and extract identity information. Thereon, the extracted identity information is cross-verified with the identity database (155).

The identity database (155) may pertain to a unique ledger of identities provided by a system for authentication of medical certificates using a distributed ledger (100), or it may even be a ledger of identity information availed from governmental portals like the NHS.

Additionally, the user module (170) includes a processing unit (171), a scanner (172), a network module (173), and a user interface (174). The user module (170) is part of the system for authentication of medical certificates using a distributed ledger (100) which exists at the user's end. The person wanting to generate, acquire or authenticate the medical certificate may interact with the system via the user module (170), and specifically the user interface (174) of the user module (170).

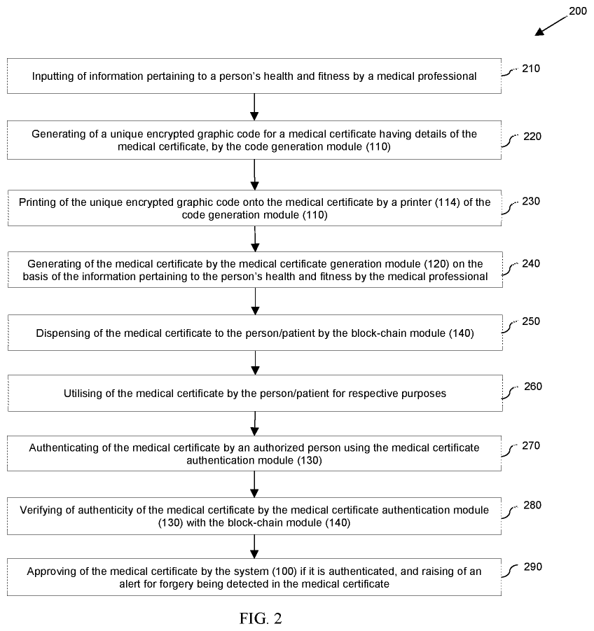

Referring to FIG. 2 illustrates a process flow diagram depicting a method for authentication of medical certificates using a distributed ledger (200), in accordance with an embodiment of the present disclosure. Furthermore, the method for authentication of medical certificates using a distributed ledger (200) comprises of multiple steps as recited below.

At step 210, the medical professional inputs all information pertaining to a person's health and fitness onto the information module (121) of the medical certificate generation module (120), via the input module (125). The information pertaining to a person's health and fitness may include information including but not limited to medical professional/examiner details and information, patient/person health and fitness information, and information regarding the ailment being experienced by the person/patient.

At step 220, the code generation module (110) generates the unique code, which may be a unique encrypted graphic code for the medical certificate having details of the medical certificate. At step 230, the unique code is printed on the medical certificate by the printer (114) of the code generation module (110).

At step 240, the medical certificate generation module (120) generates the medical certificate on the basis of the information pertaining to the health and fitness of the person/patient provided by the medical professional.

Further, at step 250, the block-chain module (140) dispenses the medical certificate to the person/patient. At step 260, the person/patient utilises the medical certificate for their respective purpose. At step 270, the medical certificate is authenticated by an authorised person using the medical certificate authentication module (130) by scanning the unique code placed on the medical certificate.

At step 280, the block-chain module (140) verifies the authenticity of the medical certificate by obtaining the information stored pertaining to the unique code in the distributed ledger database (141) and the plurality of nodes (142a, 142b, 142c, . . . , 142n).

Finally, at step 290, the medical certificate is approved by the system for authentication of medical certificates using a distributed ledger (100) if the medical certificate is authentic, and if the medical certificate is found to have forgery and be unauthentic, an alert is raised to the authorities.

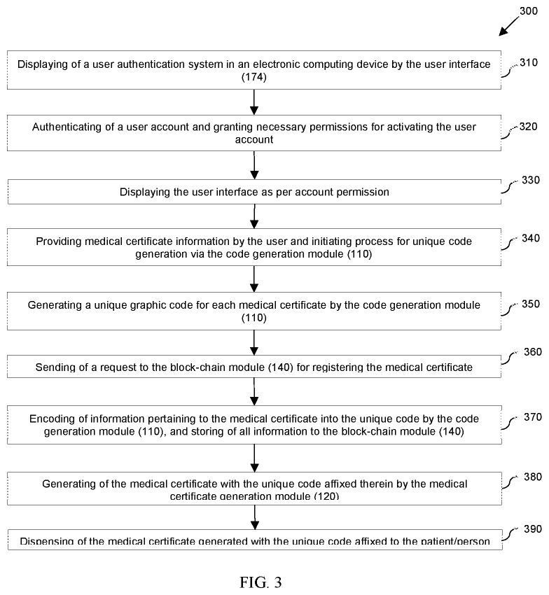

Referring to FIG. 3 illustrates a process flow diagram depicting a method for generating unique codes for medical certificates using a distributed ledger (300), in accordance with an embodiment of the present disclosure. Furthermore, the method for generating unique codes for medical certificates using a distributed ledger (300) comprises of multiple steps as recited below.

At step 310, the user interface (174) displays the identity authentication module (150) in the electronic computing device. Once the identity of the user is confirmed, the identity authentication module (150), at step 320, authenticates the user account and grants necessary permissions for activating the user account.

At step 330, the user interface (174) is displayed as per account permissions granted. At step 340, the user provides medical certificate information and initiates a process for unique code generation. At step 350, the code generation module (110) generates a unique identification number for each medical certificate.

At step 360, the medical certificate generation module (120) sends a request to the block-chain module (140) for registering a medical certificate in the distributed ledger database (141). At step 370, the code generation module (110) encodes all information pertaining to the medical certificate into the unique code, and stores all of the information pertaining to the medical certificate and the unique code onto the block-chain module (140).

At step 380, medical certificate generation module (120) generates the medical certificate with the unique code affixed therein. And at step 390, the medical certificate with the unique code affixed therein is dispensed to the patient/person by the system for authentication of medical certificates using a distributed ledger (100).

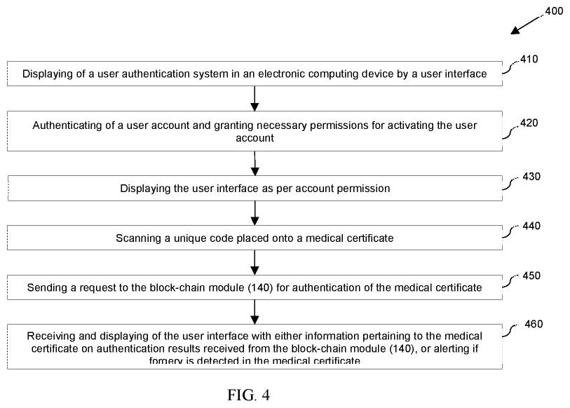

Referring to FIG. 4 illustrates a process flow diagram depicting a method for authentication of medical certificates (400) by the system for authentication of medical certificates using a distributed ledger (100), in accordance with an embodiment of the present disclosure. Furthermore, the method for authentication of medical certificates (400) comprises of multiple steps as recited below.

At step 410, the user interface (174) displays identity authentication module (150) in an electronic computing device. At step 420, identity authentication module (150) authenticates a user account and grants necessary permissions for activating the user account in the user interface (174).

At step 430, the user interface (174) is displayed as per permissions provided to the user account. At step 440, the unique code (212) placed onto the medical certificate is scanned by the user using a scanner (172) of the user module (170).

At step 450, the user interface (174) sends a request to the block-chain module (140) for authentication of the medical certificate. At step 460, the user interface (174) receives and displays either information pertaining to the medical certificate on authentication results received from the block-chain module (140), or alerts the authorized personnel if forgery is detected in the medical certificate

In an aspect of the present embodiment, the system for authentication of medical certificates using a distributed ledger (100), and the method for authentication of medical certificates using a distributed ledger (200), the identity information of a person accessing the medical certificate, generating a medical certificate, or authenticating a medical certificate, as captured by the identity authentication module (150) are stored onto the distributed ledger database (141) of the block-chain module (140) for future reference.

In another aspect of the present embodiment, the system for authentication of medical certificates using a distributed ledger (100), and the method for authentication of medical certificates using a distributed ledger (200), the medical certificates generated by the medical certificate generation module (120) may have additional authenticating descriptors beside the unique code, including but not limited to watermarks, security threads, latent images of graphics or numerals, distinctive bleed lines, and holographic marks.

In yet another aspect of the present embodiment, the system for authentication of medical certificates using a distributed ledger (100), and the method for authentication of medical certificates using a distributed ledger (200), the unique codes for the medical certificates may be generated by one or more than one authorized person or institutions only.

In yet another aspect of the present embodiment, the system for authentication of medical certificates using a distributed ledger (100), and the method for authentication of medical certificates using a distributed ledger (200), the system for authentication of medical certificates using a distributed ledger (100) may be accessed on a virtual as well as a physical platform, with back-end integration of the block-chain module (140). The physical platforms may include but not be limited to an application developed for a mobile device or computer, a website, a calling service, or a program available on specifically manufactured devices.

In an aspect of the present embodiment, the system for authentication of medical certificates using a distributed ledger (100), and the method for authentication of medical certificates using a distributed ledger (200) may also comprise of a platform where medical professionals, authorized personnel, and patients/other persons can connect for applying for medical certificates, availing as well as authenticating the medical certificates.

In another aspect of the present embodiment, the system for authentication of medical certificates using a distributed ledger (100), and the method for authentication of medical certificates using a distributed ledger (200) may also comprise of a wallet module facilitated in the user module (170) as an online service that allows users to make electronic transactions with other parties, bartering digital units for goods and services. Herein, the wallet module may include transactions of digital currency or nationally recognised currencies, as per the exchange rate valid at that point of time.

As will be readily apparent to a person skilled in the art, the present invention may easily be produced in other specific forms without departing from its essential composition and properties. The present embodiments should be construed as merely illustrative and non-restrictive and the scope of the present invention being indicated by the claims rather than the foregoing description, and all changes which come within therefore intended to be embraced therein. cm I/We claim:

1. A system for authentication of medical certificates using a distributed ledger (100), comprising
a code generation module (110), responsible for generating a unique code for a medical certificate, having a plurality of coded information (112), an encryptor (111), and a printer (114) communicably coupled together;
a medical certification generation module (120) responsible for generating the medical certificate, having an information module (121), an input module (125), and an intelligence module (126) communicably coupled together;
a medical certificate authentication module (130) responsible for authenticating the medical certificate, having a code reader (131) and a scanning module (132) communicably coupled together;
a block-chain module (140), having a distributed ledger database (141), and a plurality of nodes (142a, 142b, 142c, . . . 142n) communicably coupled together, wherein the block-chain module (140) is connected to the code generation module (110), the medical certificate generation module (120), and the medical certificate authentication module (130);
an identity authentication module (150) responsible for confirming the identity of a person accessing the system for authentication of medical certificates using a distributed ledger (100), having an identity module (154), an identity database (155), and an optical character recognition module (156) communicably coupled together; and
a central server (160);
wherein the system for authentication of medical certificates using a distributed ledger (100) is in communication with a user module (170) through the central server (160).

2. The system for authentication of medical certificates using a distributed ledger (100) as claimed in claim 1, wherein the system for authentication of medical certificates using a distributed ledger (100) is used for generating, receiving, and authenticating medical certificates.

3. The system for authentication of medical certificates using a distributed ledger (100) as claimed in claim 1, wherein the person's identity is authenticated by the identity authentication module (150) by way of a set of identity documents, a face match test (152), a liveliness test (153), or more than one of the set of identity documents submitted via a document module (151), the face match test (152), and the liveliness test (153).

4. The system for authentication of medical certificates using a distributed ledger (100) as claimed in claim 1, wherein the identity authentication module (150) reads the set of identity documents via the optical character recognition module (156).

5. The system for authentication of medical certificates using a distributed ledger (100) as claimed in claim 1, wherein the medical certificate is authenticated by the medical certificate authentication module (130) by reading the unique code placed therein via the code reader (131) and verifying it with the distributed ledger database (141) of the block-chain module (140).

6. A method for authentication of medical certificates using a distributed ledger (200), comprising the steps of
inputting of information pertaining to a person's health and fitness by a medical professional to an input module (125) of a medical certificate generation module (120);
generating of a unique code for a medical certificate having details of the medical certificate, by the code generation module (110);
printing of the unique code onto the medical certificate by a printer (114) of the code generation module (110);
generating of the medical certificate by the medical certificate generation module (120) on the basis of the information pertaining to the person's health and fitness provided by the medical professional to the medical certificate generation module (120);
dispensing of the medical certificate to the person by the block-chain module (140);
utilising of the medical certificate by the person/patient for respective purposes;
authenticating of the medical certificate by an authorized person using the medical certificate authentication module (130);
verifying of authenticity of the medical certificate by the medical certificate authentication module (130) with the block-chain module (140); and
approving of the medical certificate by the system for authentication of medical certificates using a distributed ledger (100) if it is authenticated, and raising of an alert if forgery is detected in the medical certificate.

7. The method for authentication of medical certificates using a distributed ledger (200) as claimed in claim 6, wherein the method for authentication of medical certificates using a distributed ledger (200) is used for generating, receiving, and authenticating medical certificates.

8. The method authentication of medical certificates using a distributed ledger (200) as claimed in claim 6, wherein the person's identity is authenticated by an identity authentication module (150) by way of a set of identity documents, a face match test (152), a liveliness test (153), or more than one of the set of identity documents submitted via a document module (151), the face match test (152), and the liveliness test (153).

9. The method authentication of medical certificates using a distributed ledger (200) as claimed in claim 6, wherein the identity authentication module (150) reads the set of identity documents via an optical character recognition module (156).

10. The method authentication of medical certificates using a distributed ledger (200) as claimed in claim 6, wherein the medical certificate is authenticated by the medical certificate authentication module (130) by reading the unique code placed therein via the code reader (131) and verifying it with the distributed ledger database (141) of the block-chain module (140).
